(19) United States
(12) Patent Application Publication
(10) Pub. No.: US 2018/0050217 A1
(43) Pub. Date: Feb. 22, 2018

(54) DEVICE FOR SELECTIVE MODIFICATION/DESTRUCTION OF ORGANIC TISSUE

(71) Applicant: Antonio Javier Moreno Castro, Granada (ES)

(72) Inventor: Antonio Javier Moreno Castro, Granada (ES)

(21) Appl. No.: 15/318,284

(22) PCT Filed: Apr. 30, 2015
(86) PCT No.: PCT/ES2015/070359

§ 371 Date: Jul. 12, 2017

(30) Foreign Application Priority Data
Jun. 11, 2014 (ES) P201400489

(51) Int. Cl. A61N 2/02; A61N 5/10

(52) U.S. Cl. CPC A61N 2/02; A61B 18/18; A61N 5/1001

(57) ABSTRACT

The invention relates to a device for selective modification/destruction of organic tissue and to the use thereof for biomedical purposes, which includes a magnetic field generator (1) housed in a Faraday cage (6), with coils/conductors and a device for controlling the electrical intensity and voltage parameters of the terminals (7). Said generator consists of a device for generating chaotic magnetic fields ( ) coils/conductors having a geometric configuration (FIG. 1) which generates chaotic magnetic field lines, characterised topologically in space by chaotic field lines, which surround the magnetic field lines contained within a magnetic torus or KAM island (2), said structure being topologically controlled in space since it is contained within the Faraday cage (6). In addition, the invention also includes one or more devices generating polarised electromagnetic frequencies (infrared, microwave or X-ray) (3, 3.1, 3.2) with focussing capacity (4), coherence with the resonant frequency and polarisation in the direction of maximum absorbance of the nanoparticles/biomolecules oriented in the KAM island (2) which will submerge the target tissue for the selective modification or destruction. The device may operate at the X-ray frequency with or without the use of nanoparticles/biomolecules, in which case the destruction of the target tissue (4) occurs by controlling, in the direction of the paths, the secondary electrons and other charged particles produced by the X-ray beam, in the target tissue (4), by modulating the magnetic field intensity of the magnetic KAM island (2) in the target tissue (4), and the direction of incidence of the magnetic KAM island with respect to the X-ray beam in the target tissue (4).

### THE OBJECT OF INVENTION

The Invention, as expressed in the statement of this descriptive Memory, refers to a device for changing/Selective destruction of Organic tissue, and the use of this device for Biomedical and industrial applications.

In particular, the object of the invention is a device of partial control of the magnetic field lines in a confined space (Faraday Cage) and the focusing of electromagnetic frequencies (Microwave, infrared, Optical, x-ray and Gamma Ray), aims at its application in the engineering field Biomedical, and more specifically in the hyperthermia Ablation and Tissue changes Through specific Activations of nanocapsules selectively in the target tissue (at Microwave Frequencies, infrared, x-ray), or partial control of the dynamics of the secondary electrons produced by the beam of x-rays and gamma rays, Passing By The Healthy Tissue and Tissue objective to Destroy.

### FIELD OF APPLICATION OF THE INVENTION

The scope of the present Invention focuses on the industry dedicated to the manufacture of Electrical and biomedical devices, in different sectors, especially in the area of hyperthermia ablation, Selective Activation of nanocapsules and Nuclear magnetic resonance.

### BACKGROUND OF THE INVENTION

Currently one of the major problems that we encounter in the Invasive Surgical medicine is the factor that has to be done for Palliative surgeries of certain pathologies. Being a Medical need in this area, the power to destroy/modify selectively and non intrusive, in most cases in which pathologies need removal or Destruction/Modification of a specific Organic tissue. In order to prevent Medical complications that may arise from the application of surgery, intrusive, or even make possible new treatment for certain diseases if current Solution, in addition to activate certain benefits Drug/Protein, due to its toxicity could not be supplied to a patient unless Activation was controlled directly in the target tissue of interest, and that current technology does not allow to perform accurately.

On the other hand therapies in experimentation is necessary in which the Activation of nanocapsules of differentiated tissues, and in experimental Therapies for genetic modification or Protein, where the vectors are modified nanocapsules, are currently with the limitations that can only be performed in vitro or in Organic tissues superficially, currently impossible to extrapolate the results obtained for the General public [18-21].

In the field of x-ray and gamma, the major problems in the implementation of x-ray mini beams/beams, is the incompatibility of Joint Operation of x-ray equipment and the equipment of nuclear magnetic resonance imaging. Another problem is the high rate of dose needed for the destruction of the tumor or tissue, affecting the Healthy tissue, its recovery and viability of Therapies. Finally, the homogenization of the Radiation on the target Tissue and the dose that produces Peak-Valley-Peak (FIG. 10) (P-V-p), depending on the shape of the collimator. The Dosimetry of Valley to preserve the essential factor as low as possible to allow the stem cells that are found in this, after the passage of the beam by Healthy tissue, capable of repopulating the area affected by the peak of the beams, and thus do not affect the irradiated tissue Significant, or irreversibly. In any case in Therapies with normal you need, is a moderate, confine and reduce the dimensions of the beam, and ultimately control the spread of Secondary electrons (dosage) produced by the beam as It passes through the Healthy tissue, and to direct and control the Trajectories of the secondary electrons produced by the beam x-ray, once in the target Tissue to damage and Destroy, to optimize the effectiveness of Radiotherapy. In the current state of the Art it is not possible [23, 26].

The objective of the present Invention is, therefore, the development of a device to solve such problems.

This device is based on the current Generation Studies chaotic Magnetic Fields [1, 11], which establishes as can have partial control of Entropy in the direction of the magnetic field Lines, by using the theories of Nonlinear Dynamics, Complexity and chaos (Kam Theory) [10]. Also based on the knowledge that we have today in the field of nuclear magnetic resonance [12], Ablation hyperthermia, radiotherapy [23-26] and nanoparticles [13-22].

The study of chaotic Magnetic Fields (CMF), dates back to the introduction of kolmogorov Arnold Moser Theorem (first presented In 1954, at the International Congress of Mathematicians), a Joint Work with his student Vladimir Arnold [10].

It's the time, by 1956, when they begin the Experimental Research of Tokamak in Institute of Atomic Energy ‘i. V. kurchatov>> of USSR Academy of Sciences. The First Tokamak consisted of a vacuum chamber with a toroidal Shape containing Hydrogen and an electrical Device for strong downloads through the State ionizaba gas plasma. A strong magnetic field Coil caused by powerful electromagnets to the confinement of the plasma temperatures I have avoided The Chasms [29]. But in May 2000, when it was announced that American Physicists have overcome one of the problems of nuclear fusion, the phenomenon is called the Edge localized modes (ELMS, or by its acronym in English) that would lead to an erosion of the Interior of the reactor, forcing replacement frequently. In an article published in May 2000 in the British Magazine Nature Physics, a team led by Todd Evans of Company, General Atomics, California, announced the discovery of a small Resonant magnetic field from the Special coils located inside the Reactor vessel, creates a “chaotic” magnetic interference the edge of the plasma that Inhibits the formation of Flow [30]. This manages to save one of the biggest problems in the nuclear fusion was the preservation of the bag containing the plasma (Tokamak). Datándose for the first time the effects of Magnetic Fields in Interaction with charged particles, such as the existence of these CMF, outside the cosmological context.

Will be back in 2007 when two Spanish Researchers J. Aguirre, D. Peralta salas, presented an article [1, 2], which show how the generation of these fields are more chaotic, easy to produce the thought (and outside the scope of nuclear fusion). Disproving the conjecture Stefanescu, in force since 1986, and stipulating the absence of Magnetic Fields with open field lines [28], and Mathematically prove the integrability of the configurations of the magnetic field Lines produced by the nonlinear Association of metallic conductors. Having been ratified These articles by other research centres 3,4,7 [], although nulamente exploited in practical applications.

The Generation of these chaotic fields are characterized by the topology of the magnetic field lines are three Topologies: periodic Field Lines, cuasiperiodicas (Kam Islands—tangles (KIS) and homoclinicos Field lines are folded on themselves) (LCC), FIG. 5, Top, Center and Bottom respectively. FIG. 6, white circles are the KIS, Black area in the LCC [1-8, 10].

Thus, we have that the field Lines periodic and quasi periodic structure, the more important the Kam Islands (FIG. 5, Center, white circles in FIG. 6), [1] for the Biomedical and interests, which is the result of the introduction of a perturbation, nonlinear, and frequency. In a planar System of Conductors, as announced by the authors of [1,2], using the above theories—1 [9-11], show that different initial conditions of the system Drivers, relative position of Li and L2, and the disturbance introduced (8), FIG. 1, that originate from different Bulls in the phase Space, FIG. 6, Centre [1-5, 10].

The Kam Theorem states that, if a system is subjected to a small nonlinear disturbance, Some Bulls will be deformed and destroyed. Those that survive are those that have a ratio of frequency (frequency of the orbits of the Vector Magnetic Field, generated by the generated by Li and L2, Irrational enough). That is to say, destroy those whose ratio of frequencies is closer to a Rational Number. So that survival and Topology in the space of the Islands Kam, and configuration of the Magnetic Lines depend on the driver and chaotic, L2 disturbance “and” (FIGS. 1-1) [5, 10].

On the other hand, the destruction of the toroids gives rise to Magnetic Field Lines homoclinicos entanglements, that Fold themselves entangled in Bundles, without a defined direction (FIG. 6, Black areas, FIG. 5, Bass) and where the Lyapunov exponents are positive (indicates degree of caoticidad the more positive, more chaotic) and therefore with Indices of directionality of the field lines with maximum entropias [1-11].

Where one of the most interesting results obtained in the Study of these Magnetic Fields is chaotic, The Dynamics of particles in them, and in particular the dynamics of charged particles in the homoclinicos entanglements, where recent studies demonstrate that depending on the initial Energy factors (intensity of CMF, and Energy of the charged Particle) they are confined to, and moderate in its Passage by the LCC [4-8,30].

On the other hand, in the biomedical Field, we have that hyperthermia can be used as to sensitize cancer cells to Chemotherapy. The degree of quimiosensibilizacion varies with the type and concentration of Drug, the type of tumor, the higher the temperature of the tumor, and the time difference between the heat delivery and chemotherapy. However, The Resistance of the Drugs, Don't seem to be suffering the heat sensitivity. In general a higher cytotoxicity of many chemotherapeutic agents, increasing the Strip temperature 40.5 to forty Third C. in general, hyperthermia With cooler temperatures increases the destruction of cancer cells with chemotherapy, but not the normal cells (i.e., chemotherapeutic side effects are minimized [18-20].

The three main nanoparticles (NPS) for Therapy by hyperthermia are gold Nanoparticles, Carbon nanotubes (CNT), Nanoparticles Superparamagnetic iron oxide (spions) where there is currently a wide range in the market of these NPS (Table 1) [19], [17, 19].

TABLE 1
List of reported Magnetic nanoparticles for application in the therapy and
hyperthermia based controlled drug administration [19].

| S. no | Type of magnetic nanoparticle | Application |
|---|---|---|
| 1. | Fe doped Au NPs | Hyperthermia-based therapy |
| 2. | γ-Fe₂O₃ | Hyperthermia-based therapy |
| | Cobalt ferrite | |
| 3. | Fe₃O₄ | Hyperthermia-based therapy |
| | Fe₃O₄-poly vinyl alcohol | |
| 4. | NiFe₂O₄ | Hyperthermia-based therapy |
| 5. | γ-Fe₂O₃ | Hyperthermia-based therapy |
| 7. | Fe₃O₄@chitosan | Hyperthermia-based therapy |
| 8. | Fe₃O₄@block copolymers | Hyperthermia-based therapy |
| 9. | Fe₃O₄-dextran stabilized | Hyperthermia-based therapy |
| | Fe₃O₄@Aminosilan | |
| 10. | Ferrite-Dextran stabilized | Hyperthermia-based therapy |
| 11. | Fe₃O₄-dextran (mono and bilayer) stabilized | Hyperthermia-based therapy |
| 12. | Fe₃O₄-lauric acid stabilized | Hyperthermia-based therapy |
| 13. | Fe₃O₄-lauric acid stabilized | Hyperthermia-based therapy |
| | MnFe₂O₄-lauric acid stabilized | |
| | CoFe₂O₄-lauric acid stabilized | |
| 14. | Fe@biscarboxyl-terminated poly(ethylene glycol) (cPEG) | Hyperthermia-based therapy |
| 15 | γ-MnₓFe_{2 - x}O₃ coated Acrypol 934 polymer | Hyperthermia-based therapy |
| 16. | FeCo@Au | Hyperthermia-based therapy |
| 17. | Fe@MgO | Hyperthermia-based therapy |
| 18. | Fe₃O₄@Si | Hyperthermia-based therapy |
| 19. | Fe₂O₃@SiO₂ | Hyperthermia-based therapy |
| 20. | FeNi@Au microdiscs | Hyperthermia-based therapy |
| 21. | Fe@Fe₃O₄ | Hyperthermia-based therapy |
| 22. | La_{0.56}(CaSr)_{0.22}MnO₃@SiO₂ | Hyperthermia-based therapy |
| 23. | Fe₃O₄@Au | Hyperthermia-based therapy |
| 24. | Magnetite cationic liposomes (MCL) | Hyperthermia-based therapy |
| 25. | Fe₃O₄-lauric acid stabilized | Hyperthermia-based therapy |
| 26. | Fe₂O₃@SiO₂ bound LHRH | Hyperthermia-based therapy |
| 27. | SPIONs bound fluorophore bimane | Hyperthermia-based Controlled drug delivery |
| 28. | Porous Fe₃O₄/Fe/SiO₂ | Hyperthermia-based Controlled drug delivery |
| 29. | Fe@SiO₂ | Hyperthermia-based Controlled drug delivery |
| 30. | poly-(N-vinyl-2-pyrrolidone) (PVP)-modified silica core@iron oxide shell | Hyperthermia-based Controlled drug delivery |
| 31. | Mg—Al layered double hydroxides (LDH) coated magnesium ferrite NPs | Hyperthermia-based Controlled drug delivery |
| 32. | Yolk-shell type nanospheres with movable cores of Au, SiO₂, Fe₃O₄. | Hyperthermia-based Controlled drug delivery |
| 33. | γ-MnₓFe_{2 - x}O₃ coated Acrypol 934 polymer | Hyperthermia-based therapy and controlled drug delivery |
| 34. | Fe₃O₄@lipid membrane (MCL, magnetite cationic liposome) | Hyperthermia-based therapy and controlled drug delivery |
| 35. | Fe@carbon nanoparticles bound polymer | Hyperthermia-based therapy and controlled drug delivery |
| 36. | Co@Au@poly(sodium styrene sulfonate)/poly(allylamine hydrochloride) | Hyperthermia-based therapy and controlled drug delivery |
| 37. | SPIONs@sensitive poly (N-isopropylacrylamide) hydrogels | Hyperthermia-based therapy and controlled drug delivery |
| 38. | Fe@Fe₃O₄ loaded 4-tetracarboxy phenyl porphyrin | Hyperthermia-based therapy and controlled drug delivery |
| 39. | Carboplatin-Fe@C-loaded chitosan | Hyperthermia-based therapy and controlled drug delivery |
| 40. | Zinc doped Iron oxide nanocrystals encapsulated mesoporous silica | Hyperthermia-based therapy and controlled drug delivery |
| 41. | MCL loaded 4-S-Cysteaminylphenol | Hyperthermia-based therapy and controlled drug delivery |

Living cells are very sensitive to temperature and the Rise of a few degrees can lead to Cell Death, has demonstrated the ability of absorption in the near infrared, microwave and x-ray of several anisotropic nanomaterials such as nanotubes, nanocapsules, nanostars, etc. That can be used for Photothermal Therapy. In Biological Systems, the nanocapsules of au are capable of producing a local increase in temperature may provide a therapeutic effect on the cancer cells that are selectively targeted by these nanocapsules of Au. Due to the large Absorption Cross sections of au nanocapsules, become absorbed PHOTONS and phonons can increase the temperature of the system. Such therapy is less invasive than chemotherapy or Surgery. The Photothermal destruction of breast cancer cells in vitro by using au nanocapsules with drugs has been demonstrated by skrabalak et al. [16]. Where Gold nanocapsules with an average length of Edge ±7 (65 nm Absorption Peak at 800 nm Wavelength), where Light penetrates deeper into the tissues. Due to the low absorption of Human Native Tissue chromophores were conjugated with anti-her2 monoclonal antibody, a, Direct Breast Cancer Cell (skbr-3) through the epidermal growth factor receptor. Then, the target cells were irradiated with laser pulses in the near infrared. By varying The Power Density, the duration of laser exposure, and the response time after irradiation, makes it possible to optimize the processing conditions to achieve the effective destruction of cancer cells. It was found that the target cells with the nanocapsules au responded immediately to the laser irradiation, and the cell damage was happening irreversibly at Laser Power densities greater than 1.6 W/cm2. The percentage of Dead cells increased with increasing exposure time [16].

Using multi-segmented metallic nanotubes, Salem et al. have proven method of non viral gene therapy. Nanotriangles is another candidate that can be used for the treatment of cancer cells. [16]

The selective delivery of Drugs is one of the most promising Directions in Development and actively in the medicinal use of Gold nanoparticles (GNP). Antitumor agents and antibiotics are the most popular Objects of the delivery destination. [20]

Proposed options using GNP conjugate antitumor agents with the following: paclitaxel, methotrexate, daunorubicin, hemcytabin, 6-mercaptopurine, dodecylcysteine, sulfa Drug, 5-fluorouracil, complexes of Platinum, Kahalalide, tamoxifen, Herceptin, doxorubicin, prospidin, etc. The Conjugation was carried out either by simple physical adsorption. The drugs on GNP or through use of a Linker as alkanethiol. The effect of conjugation of Drug and GNP was evaluated both in vitro (mainly) Models, using tumor Cell Cultures and in vivo, in Mice with induced tumors of different Natures and locations (Lewis lung carcinoma, adenocarcinoma of the pancreas, etc). In addition to the active substance, the target molecules (e.g., cetuximab) provide a better Anchor. It is also proposed to utilize multimodal Systems of delivery, where a GNP is loaded with various therapeutic agents (both Hydrophilic and Hydrophobic agents and auxiliaries, such as target molecules for photodynamic therapy, Dyes, etc. Most of the researchers indicate the High efficacy of Antitumor agents conjugated with Gold nanoparticles [20].

Antibiotics and other antibacterial agents are also considered as Objects that can be delivered by Gold nanoparticles. The possibility of producing a stable complex of vancomycin and Colloidal Gold and the effectiveness of such a complex against enteropathogens have also been demonstrated against several strains of Escherichia coli, Enterococcus faecium and Enterococcus faecalis (including vancomycin resistant strains). Similar results were obtained with a complex of ciprofloxacin nanocapsules with Gold, which showed High antibacterial activity to E.coli. The Anti Leukemia Drug 5-fluorouracil, conjugated with Colloidal Gold, has a significant antibacterial and Antifungal effect against Micrococcus luteus, Pseudomonas aeruginosa, Staphylococcus aureus, E. coli, Aspergillus fumigatus, and Niger. It should be noted that in all the cases Listed, the complexes of drugs With Gold nanoparticles were Stable. [20]

In the field of NPs in gene therapy, seems to be the ideal Strategy in Genetics, as well as the inherited diseases, gene therapy is an approach based on the introduction of Genetic Structures in the cells and the Body for Therapeutic purposes. The desired effect is achieved due to the expression of the inserted Gene or by partial or complete removal of the damaged or over expressed Gene function. Recently, attempts were made to adjust the structure and function of genes. In this case, Gold nanoparticles can act as an effective Agent to deliver genetic material in the Cytoplasm and nucleus of the cell [16, 20].

The Current Studies show that the absorption Properties of the metal nanoparticles are widely used in the common Plasmon unexpectedly is driven by the component of Magnetic dipole in the vis NIR Spectrum. The Magnetic contribution can be understood as the result of the Dissipation of Eddy Currents produced by the temporal variation of the magnetic field, which takes place at frequencies above the Plasmon Resonances and is strongly dependent on the size, morphology, and composition. The conductivity of metals are a major dominant Magnetic Absorption. Magnetic Absorption also depends on the shape of the particles and is Comparatively higher when the electric field is oriented along a direction closer Particle, thus Minimizing the degree of Electrical polarization. For example, elongated ellipsoids and flat Discs of aspect ratio, Magnetic Absorption is dominant at Wavelengths above ˜1300 nm. Under Illumination with co linear Electric and Magnetic Fields, The Magnetic Absorption reaches 90% of the total absorption (FIG. 7) [14]. It is important to control the heat deposition in the context of thermoplasmonic, and Heat dissipation through the radiated Heat Transfer [13, 14].

Carbon nanotubes, we have that the aspect ratio and the direction of Tubes can be controlled in the synthesis, optical properties can be tuned. Like Gold Nanoparticles, and also strongly absorb electromagnetic waves to generate Heat (FIGS. 8 and 9 [21]). The improved Optical Cross Section of CNT has been investigated for Photothermal Ablation of cancer cells at frequencies near infrared and soft x-ray [16,21, 15, 22].

In short, the Current Studies, we demonstrate that the Resonant absorption in the electromagnetic Frequency is dependent on the orientation of these particles in a Magnetic Field, where the resonance frequency specific NPS, is determined by factors of form, dimension and Composition of the NPS, and the Polarity and Direction of Incident electromagnetic Beam in relation to the orientation of the particle. (FIG. 7) [14] [14].

Thus the object of the invention, the device Destruction/Modification of Organic tissues (dcmf) using these chaotic Magnetic Fields, and the two conformations generated Topological Field Lines, magnetic field lines are chaotic, LCC, and lines of Magnetic Field confined in a toroidal or Island Kam, KIS. So if we Place our kis in the target tissue (TO) to modify/Destroy, and immersing in both healthy Tissue by the chaotic configuration of Magnetic Field Lines. We have a population differentiation in the orientation between the NPS, Oriented along the KIS, and those in the tissue to preserve and not affect the Healthy (ST), which should be oriented chaotically. Thereby creating a gradient in the Absorbance between particles that are between the St, with minimal Absorption into an incident Beam with the polarization in the direction of maximum Absorbance of the NPS, Oriented by the KIS, and will therefore have a maximum Absorbance in the Resonant activation, allowing her to reach, without active in the ST. Where Activation occurs by direct conversion of the energy of the Polarized beam and directed towards Success, by electromagnetic Resonant absorption of the NPS in the to, where energy absorption by these NPS, 1.6 W/cm2 shall be sufficient for Activation in TO [16].

On the other hand, we have the Ionizing Radiation, which is a Therapeutic Agent used for the treatment of malignant tumors. Given that originates is called Ionizing Radiation and Ion Energy is deposited in the cells. This energy can Destroy cancer cells or cause genetic changes which, if not repaired, resulting in cell death. Ionizing Radiation is divided into electromagnetic or corpuscular. Corpuscular Radiation are represented by electrons, Protons and neutrons. The electromagnetic PHOTONS, including x-rays and gamma rays. In clinical practice, the majority of the radiotherapeutic treatments are performed through the use of PHOTONS [26].

Ionizing Radiation acting on the genetic material of Cells (deoxyribonucleic acid or DNA) leading to Cell death or the loss of reproductive capacity, damage to DNA can be single or double chain. There are two Effects: (1) the direct effect of radiation damage caused by Direct interaction with DNA; (2) indirect effect on DNA: the generation of Free Radicals, arising from the ionization of water and stabilized by Oxygen [26].

Radiation therapy is an important component of the treatment against cancer. Approximately 50% of all cancer patients receive Radiation Therapy during the course of the disease. Radiation damage to normal cells and cancer cells. This is why the constant development of Therapies based on the selective destruction of radiant Tum oral cells, trying to preserve normal cells [26].

The Biological Effectiveness of the Radiation (which can be measured, for example, as the ability to produce cell death depends on the linear Energy Transfer (LET), total dose, the rate of fractionation and Radiosensitivity of target cells or tissues and the Biological effect considered. The Let is a measure of the density of ionizations produced when Radiation interacts with the tissue. The greater the let, the greater the amount of Ionization and therefore the greater the Damage [26].

The frequency of x-rays [23-26], techniques of Radiotherapy will be optimized by applying chaotic Magnetic Fields, whether in the target Tissue to destroy (TO) have a Magnetic Field, confined and localized, i.e. was bathed in our minihaz/KIS, perpendicular to the incident beam of x-rays, we will this volume, Uniform Radiation deposited by the secondary electrons produced in the tissue, the passage of the x-ray Beam This can be controlled by the magnetic field intensity and therefore to partially control the waterfalls of electrons produced by the beam to its step in tissue volume, and the desired shape, once Move or change the direction of incidence of the KIS, with respect to the x-ray Beam [31]. On the other hand we have the dynamics of the electrons produced by the passage of the x-ray Beam to pass through the St. And therefore will be bathed by the Magnetic homoclinicos entanglements, the LCC, CMF. We have identified that there are two Dynamic according to the dynamic conditions and initial Field, these Trajectories are regular, and cuasiperiodicas Electronic orbits, and therefore Confined Areas (Capture) and the other Dynamic dispersion would be identified through helical type chaotic Trajectories [5,8]. Where the confinement of particles is given by the intensity of the applied Magnetic Field, and the energy of the emitted particles. Therefore the step of charged particles by the LCC can be shortened, confinándose in its Trajectory, The Peaks (FIG. 10 [25]), to moderate their Energy. This being valid for any type of Polarized Beam with you as normal. Thus we get an extra benefit of CMF, moderating the diffusion of electrons produced by the beam to its Passage in the St. therefore Further Reducing dosimetries Valley (FIG. 10 [25]) in the tissue in which it is desirable and necessary to minimise the dose everything [23,25]. On the other hand, the biocompatibility of Issuers of x-ray beams and dcmf, no longer will be the incompatibility of operation currently owns the nuclear magnetic resonance imaging (MRI) next to the radiotherapy techniques, given the two Topologies of Field lines in the CMF, the tangles homoclinicos containment, and their ability to solve the problem of compatibility of both Technologies. Since, unlike MRI, in which the secondary electrons, the step of the beam, expanded and increased the Let The Healthy tissue (and thus the Biological damage). Dcmf was confined in these secondary electrons in the beam Path of making viable the use of Radiotherapy techniques current along the dcmf without incurring Greater Biological damage to Healthy Tissue [26].

Thus, All the current techniques of Radiotherapy will be optimized by Minimizing the Biological damage to its minimum Expression to the passage of the x-ray Beam by Healthy tissue, and channeling/directing the charged particles produced by the passage of the x-ray beam on the target Tissue to desired volume (within the constraints of restraint the charged particles produced), and thus expand the area to treat. Thus a matrix with only two Beams (with widths of 0.6 mm) may cover several volumes of the Order of cm (˜6 cm) tissue to treat (dependent on the beam Energy and Environment) and, in the final analysis, optimizing results with current techniques, which can not be achieved.

However, as a reference to the current state of the Art, at least on the part of the applicant, is not aware of any device or Invention to present a technical and Structural Characteristics similar to those presented here for Biomedical Device use is advocated, as claimed.

### EXPLANATION OF THE INVENTION

The device Modification/Selective destruction of Organic tissues suggests that the present Invention is configured as a Novelty within its field of application, and under whose implementation is achieved satisfactorily the objectives outlined above, and the characteristic and details that make it possible to distinguish it from what is already known in the conveniently collected final demands that accompany this descriptive Report of the same.

In particular, for the establishment of the proposed scheme is essentially creates a distribution of Conductors (FIG. 1, having countless viable Geometric conformations of Drivers [1]—3.10) in such a way that allows us to generate a Magnetic Field (CMF) (0.01 g-20 t chaotic), the passage of an electric current modulation. The Magnetic Field will be formed by chaotic Field Lines homoclinicas Tangled Magnetic Field Lines, i.e. chaotic (LCC), and A/number/s of toroid Magnetic or magnetic Islands Kam, Kam (KIS), formed by lines of Magnetic Field confined, all with the same direction, both Topologies of Field lines in a controlled manner and Confined Space, in a Faraday Cage. Through techniques known and described in references [1]—11-5.7. Where the control of the CMF Theory apply especially Kam [10], which sets as these Islands are determined by Kam, a small disturbance morphological non-linear, one of our drivers (Linear). Where the existence and survival of the quasi periods of stable Magnetic Field, KIS, we determine the theories Kam-3.10 [1], and in particular for the configuration of Conductors of FIG. 1 Is determined by the position of the driver and the perturbative amplitude L2 “and” [1,2,5].

Where mechanically speaking, the device Destruction/Modification of Organic tissues remotely (dcmf), operates on the same principle, Mechanical and electronic, magnetic resonance imaging (MRI) [12]. Where It generates a Magnetic Field confined in a ring, like you would in a CPM in the kis of the MRI, which applies a current driver for a ring, which is the Simplest configuration for generating a Magnetic Field [12]. Giving rise to a generation of Magnetic configuration in space with all lines of Magnetic Field aligned along the Axis of the Ring. This device takes a step forward in the technical, generating a Magnetic Field chaotic, characterized by two Topologies on the space of Field Lines, as it was no longer needed. Where a Ring conductor confined, as currently happens in NMR, but these Topological structures of KIS, will be underpinned and Enveloped by the magnetic field lines are chaotic, LCC.

On the other hand, in the field of Nanoparticles (NPS)/nanocapsules, these are characterized by their Resonant Absorbance of anisotropic, under the guidance of these in a Magnetic Field, to the front of the electromagnetic beam and the direction of polarization of the beam in space with respect to these NPS (FIGS. 7 [14], 8 [21] and 9 [13], 14,21,22). Where the frequency characteristic of Resonance Of these nanoparticles is determined by factors of shape and Dimensions of the NPS. The mechanisms of Absorbance and Resonant Frequency are determined by the characteristic Absorbance that occur directly or by the surface Plasmon resonant (Surface Plasmon resonance (SPR) of the NPS (Frequency Optical frequencies in the visible, infrared and Microwave) [13,14] or Resonant Absorption by electrons On The Orbit, The Energy Transfer orbits of S-d or S—[21,22] (or the optical frequencies of x-rays), or by removal of the Resonant vibrational or rotational moments (The Optical frequencies of infrared and Microwave). Thus the Activation mechanisms of these NPS usually depend on the Absorbance of these Resonant, and therefore these are the spatial orientation with respect to a Wave Front (Coherent electromagnetic Resonant Frequency) and Polarization in the space of this Wave front with respect to the orientation of the direction the NPS in a Magnetic Field [13,14,21,22].

The applicability of a CMF in Space, we provide Biomedical levels, the possibility of creating a gradient controlled, in the directions of these NPS. Therefore a gradient controlled the Absorbance in the Resonant frequencies of NPS. Always have the magnetic susceptibility (X) and anisotropies in the appropriate Absorbance [13,14,21,22].

The guidance addresses of these NPS embedded in the tissue, immersed in a CMF is determined whether these NPS are in the Topological Space formed by the magnetic field Lines LCC or Kis. Taking the NPS that are localized in the Topological Space formed by the chaotic Field Lines, their Spatial orientation will be chaotic, Random. On the other hand the particles located in the Topological Space of the KIS, “todasi” will be oriented along the direction of KIS, Resonant Absorbance with a well-defined Space.

What a time you Inject a solution of NPS (dependent pathology or modification is desired), an organic tissue, bathed by a CPM. The selective Activation of these NPS will come under the direction of these NPS and the incidence of the optical Beam in the middle, which must have the polarization and Direction of these NPS in the kis is Resonant Absorbance of these NPS. The frequency of the beam (as Coherent as possible according to the technique, The Frequency should be the specific resonance (Microwave, infrared, Optical or x-ray) of the NPs introduced [13,14,21,22]. With what will be differentiated the absorbances of these NPS in Optical Resonant Frequencies, depending on whether these NPS are submerged by the LCC, and therefore oriented chaotically Thus, with a minimum capacity of Absorbance to Beam with the Resonant frequency of these NPS given the Anisotropy in the absorbacia applicable of these NPS. Or if these are submerged by the magnetic field formed by the KIS, and therefore “All” oriented and aligned in the direction of the KIS, allowing the occurrence of a Polarized Beam, directed in the direction of maximum Absorbance, the NPS, and the resonance frequency of the specific NPS. This would allow Direct deposits of Energy on the target tissue (TO), or Activations of these NPS in the to, through the conversion of the electromagnetic energy of the incident Beam, Resonant and Optimum way on all these specific NPS. Where the Activations may be produced from energy deposited in the 1.6 w/cm2 TO [16]. This allows a selective Modification and Destruction/differentiated once to confine and get our, our Magnetic toroid or KIS, characterized and localized in Space.

The Absorbance and differentiated between TO and ST, will depend on the number of NPS introduced, and of these, which are oriented in our success. According to the statistics of the intensity of boltzmanni, CMF, hcmf susceptibilities, Magnetic Nanoparticles, NPS, and the rotational Energy (Temperature dependent (Middle), could affect the viscosity of the medium to low intensities of CMF). At sufficiently High intensities greater than 0, (|5 t, hcmf −% NPS|<<k T, K—Boltzmann Constant, T—medium Temperature), we can assume that all will be oriented in the direction of the kis [32], regardless of the XNPS of the main NPS.

Where the frequencies of the x-rays and gamma rays, have other mechanisms for the destruction of the to, through implementation and Generation of CMF.

If we consider that the Technical minihaces/x-ray beams, and Beam Forming, produced by a collimator, FIG. 10, [25] Apply Polarized beams, instead of normal, we have that the Photoelectron Emission by photoelectric effect is not isotropic. At Low Energy, whereas a non Relativistic and semi-classic, the electron would be ejected from the Atom due to the electric field of the electromagnetic Wave that acts on him. The probability of being emitted in a certain direction is proportional to the square of the projection of the Electric vector of the Wave On the direction of the initial velocity of the Electron, and hence the transfer time of the particles will be in the direction of polarization of the Beam.

On the other hand, the Compton effect would have a similar situation, Emission and dispersion in a dimension to be Polarized Beam. This would in our Benefit when the Radiation Dosimetry of the P-V-P, FIG. 10, [25] reduce lateral Radiation produced by the Compton and photoelectric effects in tissue, Safeguard, St. However there will always be a remnant Dispersions produced by the emission of electrons and iterations due to Ionization Ionization Tertiary (Auger electrons, and electrons produced by Ionization Secondary Ionization and absorption of PHOTONS scattered by the main Beam, The Energy produced by the secondary electrons [26].

If the target Tissue to destroy (TO) we apply a Magnetic Field confined and localized, i.e. was bathed in our minihaz/KIS, perpendicular to the incident x-ray Beam, enable us in this volume, Uniform Radiation deposited by the secondary electrons produced in the tissue, the passage of the x-ray Beam/gamma This can be controlled by the magnetic field intensity and therefore to partially control the waterfalls of electrons produced by the beam to its step in tissue volume, and the desired Shape [31]. For a lookev incident Radiation, Kinetic Energies of electrons emitted from 10 keV to lookev, thereby to obtain Radii of curvature (R) of LMM, we Field intensities 0 It.33 t respectively, for G=1, IF G>1, R. although it will be the transfer of effective time in this direction, perpendicular to the direction of the speed, and thus optimizing the dosage of Radiation in the target tissue. Where Radiation beams with normal (MeV), the mean free Path is of the Order of cm (8 mev˜4G/cm2 rcsda (Continuous Slowing down approximation, Berger and Selzer 1983, Table 2) [27]. Therefore the introduction of a confined Magnetic Field, perpendicular to the beam path would provide Greater Dosimetry in the Valley to larger Separation distances (dependent on the beam Energy and the intensity of the CMF).

The application of Magnetic Field also creating new mechanisms for introducing a higher linear Energy Transfer (LET), in the to, dependent on the intensity of the magnetic field, CMF, this being valid also for minihaces/do not Polarized. Where control of the intensity and Polarity of the magnetic field will provide us to make better use of our minihaz/x-ray Beam. From table 2, in the range of MeV and considering the Polarity reversal of the magnetic field applied, this will enable us to address the secondary electrons in one direction and its opposite, thereby Doubling the effective scope of the therapy, which will enable us to handle Greater volumes, partially Controlling the Trajectories of the secondary electrons produced by The Modulation of our cmf [31], the desired Volume to To provide a solution to the problems that demand and the techniques of today do not allow [26].

TABLE 2
CSDA (Continuous slowing down approximation) distances calculated
with the ESTAR program [43]

| | |
|---|---|
| Energia (E_{d}) | Alcance g/cm², Agua |

MeV

| | |
|---|---|
| 0.01 | 0.000252 |
| 0.02 | 0.0009 |
| 0.04 | 0.0029 |
| 0.08 | 0.0098 |
| 0.1 | 0.0143 |
| 0.2 | 0.0449 |
| 0.4 | 0.1282 |
| 0.5 | 0.177 |
| 1 | 0.4359 |
| 2 | 0.98 |
| 5 | 2.55 |
| 10 | 4.98 |
| 20 | 9.32 |

On the other hand we have the dynamics of the electrons produced by the passage of the x-ray beam as It passes through St. and therefore will be bathed by the Magnetic homoclinicos entanglements, the LCC, CMF. We have identified that there are two Dynamic according to the dynamic conditions and initial Field, these Trajectories are regular, and cuasiperiodicas Electronic orbits, and therefore Confined Areas (Capture) And the other Dynamic dispersion would be identified through helical type chaotic Trajectories [5,8].

Considering the dynamics of the Electron Beam generated by the Ionizing x-rays, in a dense medium, and given the capacity of confinement of the LCC will be affected its path length. With this scope will be shortened, confinándose Peaks in its trajectory (FIG. 10 [25]) to moderate their Energy. This being valid for any type of Polarized Beam with you as normal. Thus we get an extra benefit of CMF, moderating the diffusion of electrons produced by the beam to its Passage in the St. therefore Further Reducing dosimetries Valley (FIG. 10 [25]) in the tissue in which it is desirable and necessary to minimise the dose everything possible. So we can use these entanglements homoclinicos Field, the techniques of x-ray Beam we trace the Path Towards Success, through our CMF, such that the sum of the Lyapunov exponent in the field in your career, caoticidad (Index of the Magnetic Field [1-11 maximum]), thus guaranteeing the greatest effect of confinement of the Field to Field or Beam Energies of CPM determined [5].

Where the details of the dcmf come with conformations of x-ray Beam, as treated by the technique of radiation therapy for minihaces (minibeam Radiation Therapy) [23-26] FIG. 10 [25], that is doing with a high degree of Coherence, i.e. collimated by matrices Millimeter, Wide Beam only 0.5 mm and separation between the beams of 1.4 mm or larger, but applying it with higher Beam Energies, of the Order of MeV, which is a matrix with only two Beams (with widths of 0.5 mm), and therefore damage Healthy Tissue only 0.52 mm. May cover several volumes of the Order of cm (˜6 cm3) tissue to treat (dependent on the Energy and Beam). Thus we have, for the destruction of the already not to be restricted, this is directly dependent on the effective section of the Beam.

On the other hand, the biocompatibility of Issuers of x-ray beams and dcmf, no longer will be the incompatibility of operation currently owns the nuclear magnetic resonance imaging (MRI) next to the radiotherapy techniques, given the two Topologies of Field lines in the CMF, the tangles homoclinicos containment, and their ability to solve the problem of compatibility of both Technologies. Since, unlike MRI, in which the secondary electrons, the step of the beam, expanded and increased the Let The Healthy tissue (and thus the Biological damage). Dcmf was confined in these secondary electrons in the beam Path of making viable the use of Radiotherapy techniques current along the dcmf without incurring Greater Biological damage to Healthy tissue.

The Precision of the Destruction/Modification of tissue is determined by the control over the Islands Kam our CMF, which will be marked and well localized in Space [1]—5:10. So that the Topological Control Area for Destruction shall be determined:
The impact of effective diameter or section of our beam of electromagnetic frequencies on the target region, TO.
Intersection of our beam of electromagnetic frequencies with the Island Kam to apply on the target tissue. Where the diameter of these Islands may be controlled by applying theories Kam [10], in particular for the configuration of Conductors of FIG. 1, the amplitude parameter “and” L2.

To optimize the use of CMF will provide a Vision System, to focus on the area of Destruction with precision. The level of current technology, provides us with the technology of nuclear magnetic resonance (NMR) [12].

In current techniques of nuclear magnetic resonance [12], we have as our driver System (FIG. 1), it generates a Magnetic Field confined, reduced and well localized, as we have in our system, i.e. the Islands Centre, Kam (FIG. 5, FIG. 6 white circles [1]). Therefore, the knowledge that we have today in the technology of nuclear magnetic resonance imaging (MRI) may be applied to dcmf. As in the MRI, Magnetic Fields are confined, KIS, to guide the nuclear Magnetic Moments. The techniques make use of MRI for the orientation of these moments of confined nuclear Magnetic Fields generated by the Simpler configuration of Conductors, i.e. through the use of circular coils. With a Magnetic Field chaotic is a step ahead in Technology, because we have Partial control of the magnetic field Lines or not, chaotic, LCC, KIS, in a confined space with a configuration of Conductors, something more complex, FIG. 1, but without the need to wrap the magnetic field, KIS, a driver, as is currently done. The design of this device Generator must provide a range of optical frequencies to cover nuclear RF Frequency (MHz) [12], and operate as a nuclear Magnetic Resonance. The current use of MRI gradient coils, to locate the point of viewing [12], which in this design would not be necessary, a priori. By applying the knowledge of the final MRI will allow the dcmf See with the same precision which is the target tissue.

Other advantages and possible applications of the device include:
new models of Open magnetic resonance imaging (MRI, MRI).
control in the entropias Ionic mixtures.
compatibility of equipment emitting x-rays and Nuclear magnetic resonance.
improvement in the Dosimetry in the Boron Neutron Capture Therapy.

### BRIEF DESCRIPTION OF THE FIGURES

FIG. 1 shows an example of the geometric configuration of Drivers/Superconducting coils to generate Magnetic Fields Applied chaotic, once a Flow of Electrical current. Note that although this configuration of Conductors, has the ability to produce a CMF, the passage of an electric Current, characterized by the generation of chaotic Magnetic Field Lines, Magnetic homoclinicos entanglements LCC, and lines of Magnetic Field confined in a toroidal Magnetic or Magnetic Island Kam, well characterized Topologically in Space (FIG. 5, FIG. 6 white circles) [1-5, 10]. The configurations of Drivers with a Generating capacity of CMF as reported in Literature [1-5, 10], are innumerable, which, depending on Industrial needs The configuration and conformation of these drivers capable of generating a magnetic field could be chaotic. Mathematically is described in cylindrical coordinates by: LI={X=0, y=0}, L2={X=cos (c)=[) Sen (CB), Sen (Z=8 K (c)).

FIG. 2 shows a schematic Block Diagram as a representative example, Device Modification/Selective destruction of Organic tissues, the object of the invention, which are the main parts and Elements it contains. Preferably operating at the frequency of X Rays.

FIG. 3 shows a schematic Block Diagram as a representative example, Device Modification/Selective destruction of Organic tissues, the object of the invention, which are the main parts and Elements it contains. Preferably operating at the frequency of infrared and Microwave.

FIG. 4 shows a schematic Block Diagram as a representative example, Device Modification/Selective destruction of Organic tissues, the object of the invention, which are the main parts and Elements it contains. Preferably operating as Magnetic Resonance Imaging and dcmf.

FIG. 5.—Graph of the three types of Magnetic Field lines in a Magnetic Field generated in the perturbed chaotic System, FIG. 1 (b), where Epsilon=0.05. (top) Elliptic periodic Magnetic line. (Center) cuasiperiodicas Stable Lines or Islands Kam, Kis. (bottom) homoclinicos Tangled Magnetic Field Lines or chaotic, LCC [1]

FIG. 6.—PMC Poincare section of FIG. 1 (a), parties are the White Islands dotted Black area Kam Magnetic, Magnetic homoclinicos entanglements, FIG. 6, Center and Bottom respectively, for Epsilon=0.05, 0.02) and B=[1].

FIG. 7—Spectral Dependence of the Magnetic contribution to the absorption of Gold nanoparticles of different shapes and aspect ratio: (A, D, g) Ellipsoids, (B, e, H) Records, and (C, F, i) Rings (a)-(c) effective cross section absorption of Incident electric field and two different orientations with respect to the Symmetry Axis Magnetic particles (see insets in (a) and different Particle sizes (see insets above) (d)-(i) fraction of losses in Magnetic, Solid curves (d)-(i) are obtained using a Drude Model permittivity for Gold, while the rest of the calculations are tabulated using Optical Data. In the directions Parallel and perpendicular polarization Components refers to the direction of K [14].

FIG. 8.—in the box on the Upper left illustrates the electric field vector at different polarizations between 0o and 90o. The points are the relative proportion of 71 (conduction Band) or absorption (conduction Band) measured at 60 horizontal Angle to the Axis of alignment (see FIG. 9). The Continuous Line is obtained from the simulation of propagation with Gaussian with Standard deviation of diameter 27 8 . The Box in the lower right part represents the angular Dependence of the Absorbance and a service with respect to the nanotube AXIS (horizontal dashed line) [21].

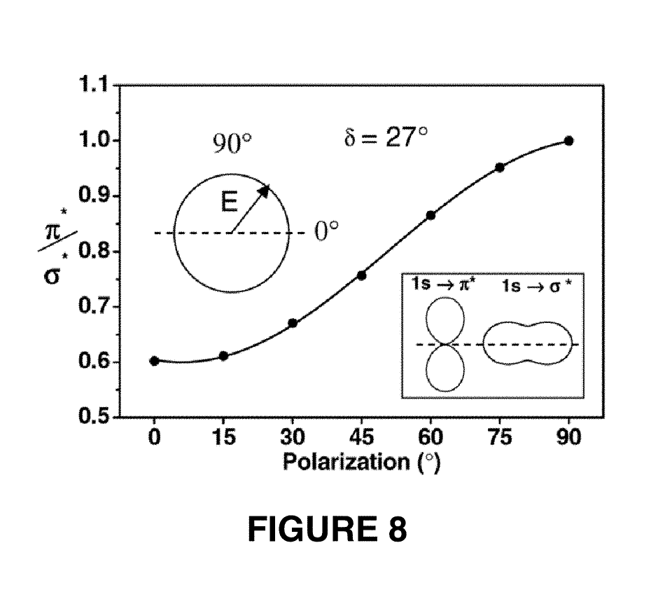

FIG. 9.—x-ray Absorption Response of vertically aligned SWNTs. The Angle Between the incident beam and the Axis of alignment is 60th in the horizontal plane. The linear polarization of Light Moves from horizontal to vertical (0) (90). The Spectra are Scaled to the level of absorption (or orbital Electronic) [21].

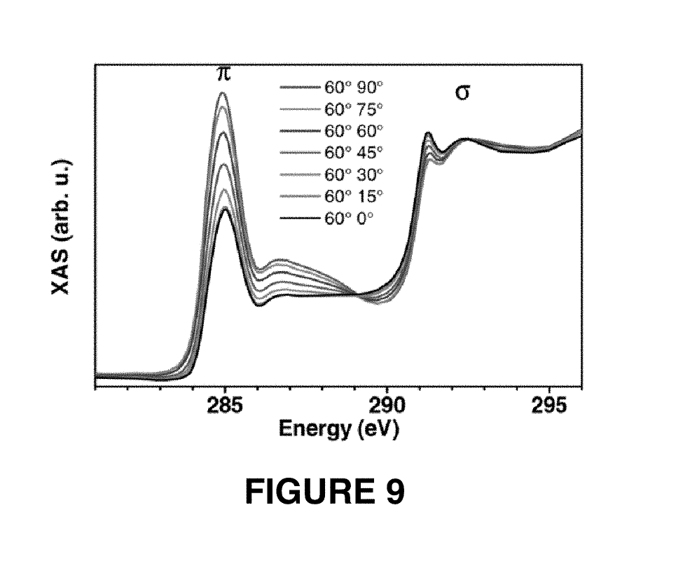

FIG. 10: Schematic of the Geometry of COLLIMATION and Beam Dosimetry used in micro/minirayos MicroBEAM Radiation Therapy [25].

### PREFERRED EMBODIMENT OF THE INVENTION

In view of these figures, and in accordance with the numbering adopted, May be seen in a non limiting example Device Destruction/Selective Modification of Organic tissues advocated, which includes the parts and elements that are identified and described in detail below.

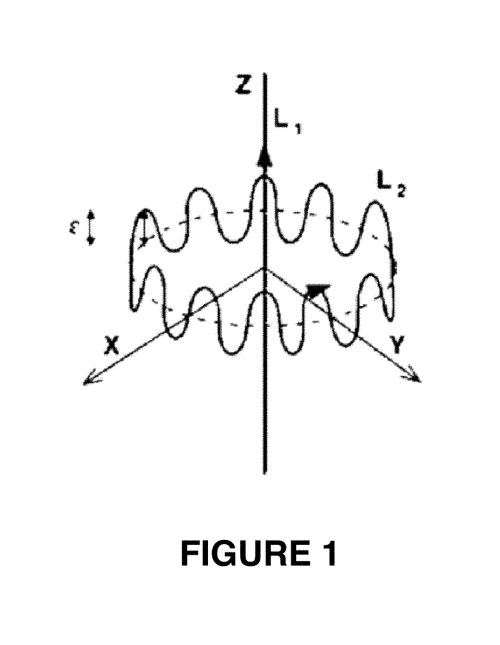

FIG. 1 shows a Configuration model of the configuration of Drivers/coils that the passage of an electric current to generate a Magnetic Field chaotic, although, as already reported in literature, the geometric Configurations, with capacity to generate a CMF are countless [1]—5:10. The constituent threads in FIG. 1 will be Superconducting material that will constitute the “coils”, defined as coils, but these are not will overwhelm any Cylinder if not take the geometric Shape defined by FIG. 1. These threads may consist, i.e, NB ti or NB SN in cu Matrix which are Extruded to form Threads of a Section of 0.5 mm DIAMETER. With a capacity of supports much higher than normal current drivers, 10 mA CM “-NB SN, and capable of generating Magnetic Fields up to 15 T. Other Superconducting Materials, capable of being used in the geometric configuration of Conductors, and could be nb3al mgb2.

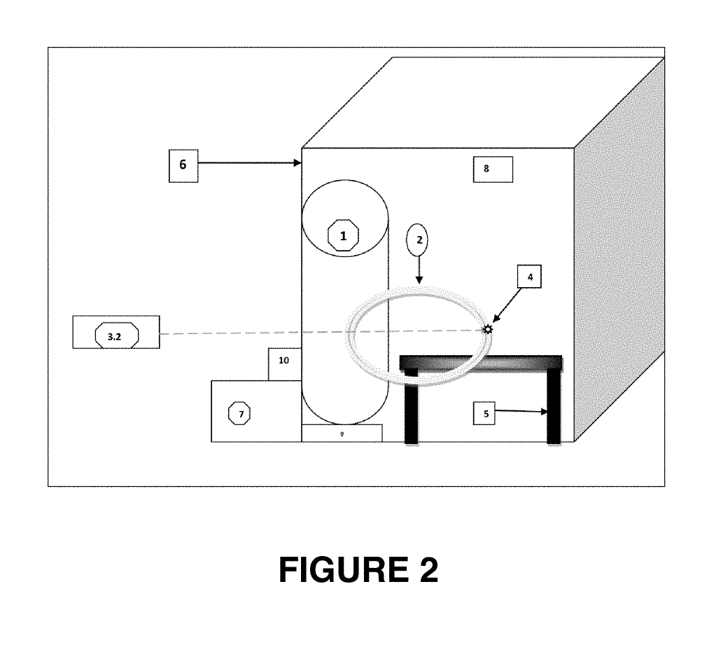

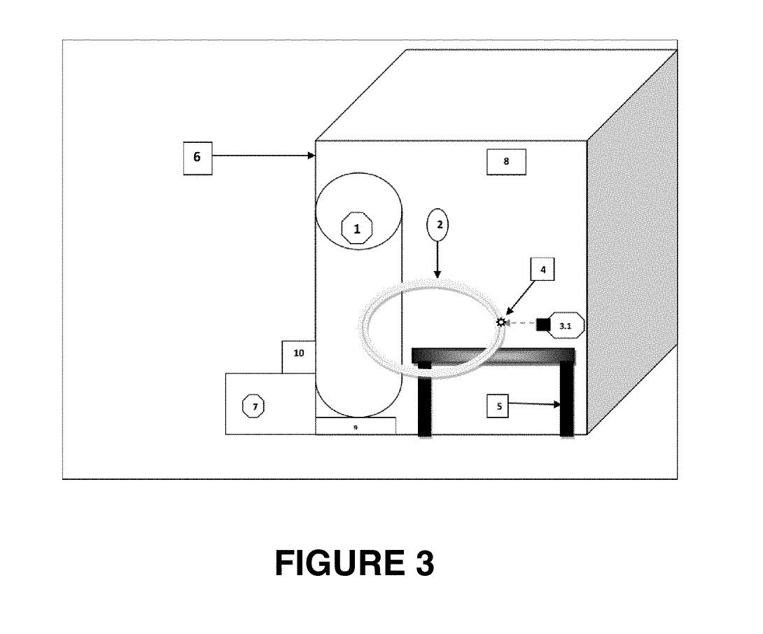

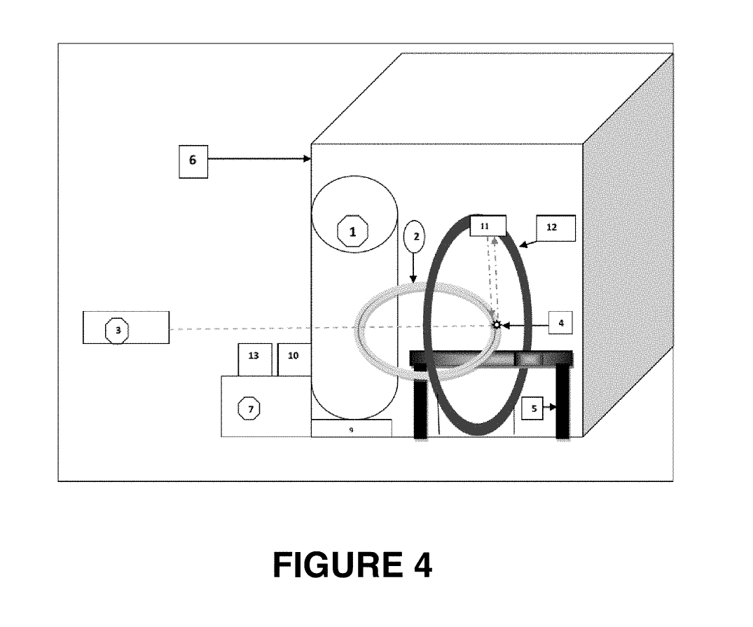

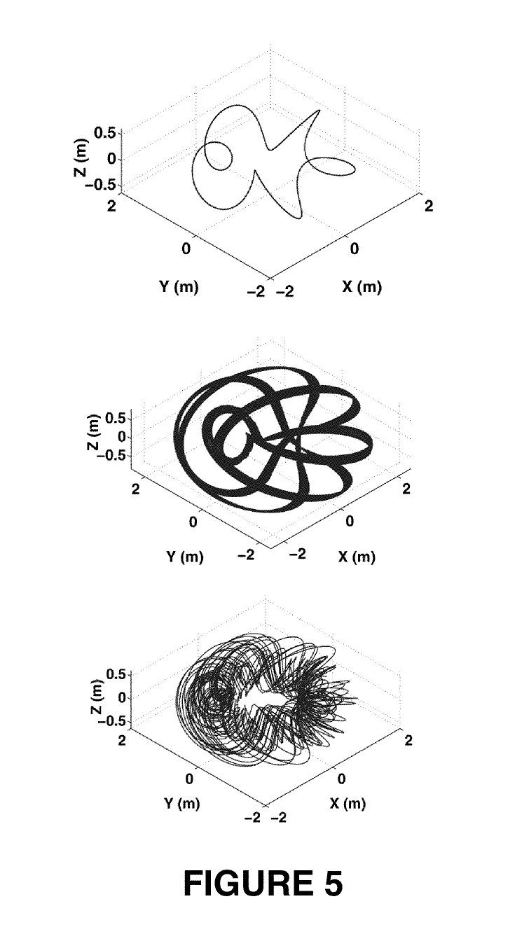

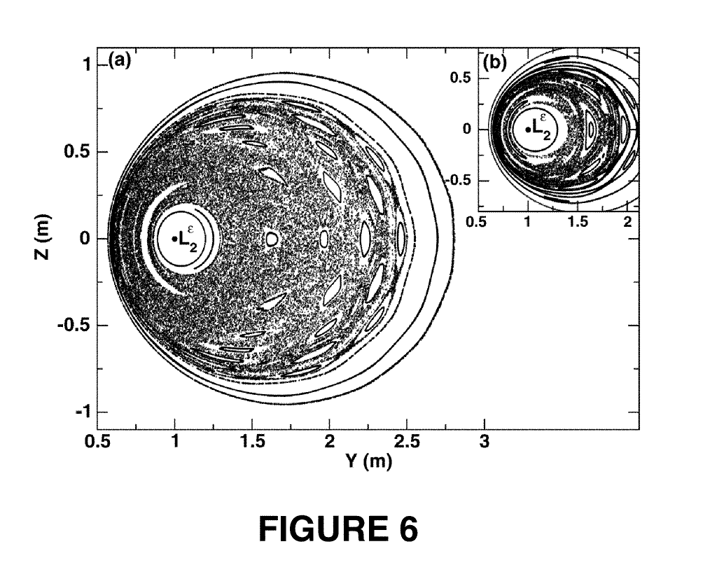

Thus, as seen in FIGS. 2, 3 and 4, which shows an example of the device in question represented disproportionately to enable a better understanding of all its parts and Elements, consists, essentially, of the following (The numbering refers to the numbering of the diagram):
1. A Chaotic Magnetic Fields Generator device, which comprises a set of coils/drivers whose Geometric configuration (FIG. 1) allows the generation of chaotic Magnetic Fields, giving rise to a number of Magnetic Islands/Kam (2) (FIG. 5 and FIG. 6 Centre, surrounded by white circles), chaotic Field Lines LCC, not shown in the diagram because they cover the whole space inside the Faraday Cage (6), where the magnetic field is confined chaotic, CMF. The cilindró Host Configuration is permeable to the Superconducting coils, Magnetic Radiation, and serve as a Barrier against accidental Direct contacts and Conduit System refrigerator coils, which is required under the operation of the Power dcmf. In the mode of operation with an x-ray device, May be fitted on the spin Plane formed by L2 (FIG. 1), and in this way, change the direction of incidence of the KIS, on the intersection with the x-ray Beam, and facilitate control “partial”, Partial to do normal and because only those “Whose Control and Direction of the velocity Component perpendicular to the applied Magnetic Field possesses KIS (2) That will be the majority) of the Trajectories of the secondary electrons produced in the to (4).
2. Magnetic Island Kam, generated by the geometrical configuration of the coils the KIS (FIG. 1, FIG. 5, FIG. 6 white circles) is Topologically characterized and localized in Space, confined by the Faraday Cage (6). It is noted that in the generation of the CMF, may have more than one Kam Magnetic Island, although for Biomedical Applications, it is only necessary to be characterized, and the beams of electromagnetic Frequency, not interseccione With those that are not the to (4).
3. Device/s Generator of electromagnetic waves, with the polarization in the direction of maximum Absorbance or Resonant Absorbance, the NPS that are oriented in the direction of the KIS (2), and Focusing and Collimating capabilities to the point/Volume TARGET (4), where the beam shall be consistent with the frequency Resonant Absorbance of NPS applied in (4) (within the constraints of the technical abilities), and Deposit Energy in the to (4) of at least 16 W/cm2. Depending on the frequency of operation and adaptation to the Shaping Device dcmf may vary:
   1. The frequency of Infrared emitters and Microwave (1014 hz -109 hz), Therapeutic Mode, the Emitter or source of the beam must be in contact with the patient (FIG. 3), to prevent the loss of Energy Beam by Reflection/Scattering Surface Wave in the Air Corps, and on the basis of targeting. Thus, the device shall be as indicated in FIG. 3 inside the Faraday Cage. Whenever Electromagnetic Compatibility between the emitting device of electromagnetic Wave, and the magnetic field Generator, chaotic, if I hadn't, may also be charged outside the Faraday Cage (6), and Channel Wave using Probes to the patient.
   2. The frequencies of the emitting X Rays and gamma rays (1016 hz-1020 hz), in this case may not operate or jointly with NPS in (4), the issuer may have capacities of polarization Beam (This is an optional criterion of Power needs, and Beam Beam Forming), which allows more consistent the market and Technical capabilities, and focus the beam down to (4). The Beam may be formed by geometrically collimators, to adapt to the minihaces x-ray Techniques [23-25].
4. Point of intersection Beam electromagnetic, Magnetic Island, Kam, and Point/Volume alteration/destruction of Organic tissues. In MRI, Point of view. The Topological Control Area for Destruction shall be determined:
   The impact of effective diameter or section of our beam of electromagnetic frequencies (dashed arrow FIG. 2,3,4) on the target region, to (4).
   Intersection of our beam of electromagnetic frequencies (dashed arrow FIG. 2,3,4) with the Magnetic Island Kam to apply on the target tissue.
5. Table or room where you will stay the patient must be permeable to Magnetic Fields, and may have a Micrometer Movement System Based on case based Industrial and Medical, more effective Shift (5), instead of the field Generator (1), and in this way move to (4), through the whole volume of the target Tissue to be treated.
6. Faraday Cage structure, which must preserve the chaotic Magnetic Field, of any kind of external electro-magnetic disturbance, given the sensitivity of these CMF to Magnetic disturbances, so that the Earth Resistance of the same will be minimal, and isolated electromagnetic leakage.
7. Control Unit from which they modify and control the electrical Parameters (intensity and Voltage terminals) of the coils/drivers housed in (L).
8. Temperature control device and for positioning the laser markers, (Volume 4), and Stereotactic operation mode.
9. Damping Device for Vibration Control and Mechanical disturbances in the magnetic field Generator (1), to avoid all kinds of Noise Source on the Field, because of its sensitivity to external conditions, comprising Said device, preferably on a Vibration Platform on which is installed the Generator of Magnetic Fields, May be charged Motor System and micrometric Displacement at The base, for moving (1) To this point, and (4).
10. Control Unit of the refrigerator System drivers/coils (1).
11. In operation, such as magnetic resonance imaging, Transmission and reception of RF coils (MHz), (10) that will House The Frequency Synthesizer, digital envelope RF Power Amplifier and antenna. In this case will be as shown in FIG. 4, Inside the Faraday Cage, Channeled along the device (11), so that we can change the point of focus, Data Collection. For the chaotic Magnetic Field Generating device (1), and shall MOVE TOGETHER synchronously for the displacement of (4) through the tissue, and formation of image.
12. Lanes for the displacement of the device (11) for the Shaping of the image must be a permeable Magnetic material to radiation.
13. Control unit and transcription Signal (11), for operation in MRI. Dashed arrow (FIG. 2,3,4 -→) electromagnetic Beam to the proper frequency of operation of the NPS (infrared, Microwave, x-ray and Gamma Ray) Dashed dotted Arrow (FIG. 4->), RF Signal emission, reception to the operability and MRI.

With all this, the operation of the device to achieve the Destruction/Selective Modification of Organic tissue, will be as follows:

Introduce a solution of Nanoparticles and biomolecules, NPS, in the patient, so that allows us to a suitable concentration for the purposes of Destruction of the target Tissue modification [17]. These NPS embedded in the tissue, immersed in a CMF, covering the volume inside the Faraday Cage (6) is determined if these NPS, housed in the tissue of a patient to treat In the Topological space are formed by the magnetic field Lines LCC or KIS (2). Taking the NPS that are localized in the Topological Space formed by the chaotic Field Lines, their Spatial orientation will be chaotic, “Random”. On the other hand the particles located in the Topological Space of the KIS (2) will be “todasi” oriented Along these KIS (2) with a well-defined direction Resonant Absorbance and characterized in Space [13,14,21,22].

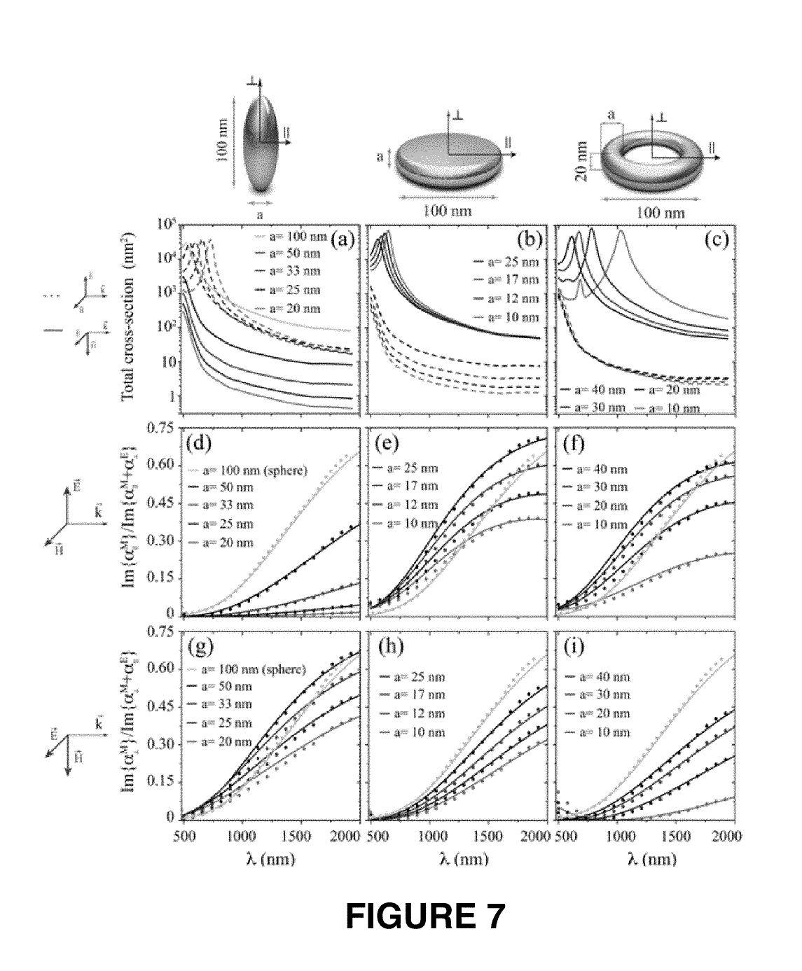

The selective Activation of Nanoparticles and biomolecules, NPS, is dependent on the direction of these NPS and the incidence of the beam optical medium (dashed arrow in FIG. 2,3,4), which must have the polarization and Direction of these NPS in the KIS (2) which is Resonant Absorbance of these NPS [13,14,21,22]. The frequency of the beam (as Coherent as possible according to the technique, The Frequency should be the specific resonance (Microwave, infrared, Optical or x-ray) introduced the NPS. With what will be differentiated the absorbances of these NPS, depending on whether these NPS are submerged by the LCC, and therefore oriented chaotically. Thus minimum Absorbance capabilities into a Beam with the Resonant frequency of these NPS due to the anisotropy of the NPS in the absorbacia applicable 14,21,22 [13]. Or if these are submerged by the magnetic field formed by the KIS (2), and therefore “All” oriented and aligned in the direction of the KIS, allowing the effect of a Beam Polarized in the direction of maximum Absorbance and the resonance frequency of the specific NPS. This would allow Direct Energy on deposits or Activations of these NPS in (4), through the conversion of the electromagnetic energy of the incident Beam, Resonant and Optimum way on all these specific NPS. Where deposits of L, 6 W/cm2 in the to (4), will be a Destruction/Selective Modification and differentiated, and once to confine our STEP (4), our Magnetic toroid or KIS, characterized and localized in Space (2). So we have a differential Absorbance between St and (4) to (between the NPS that we have introduced in the tissue and bathed by the LCC and the KIS (2)), having differences in Absorbance/Section effective absorption of Order—10000 (FIG. 7), and thus Activation of NPS, with the capacity Destruction/Selective Modification of Organic tissues non intrusive (without Surgery).

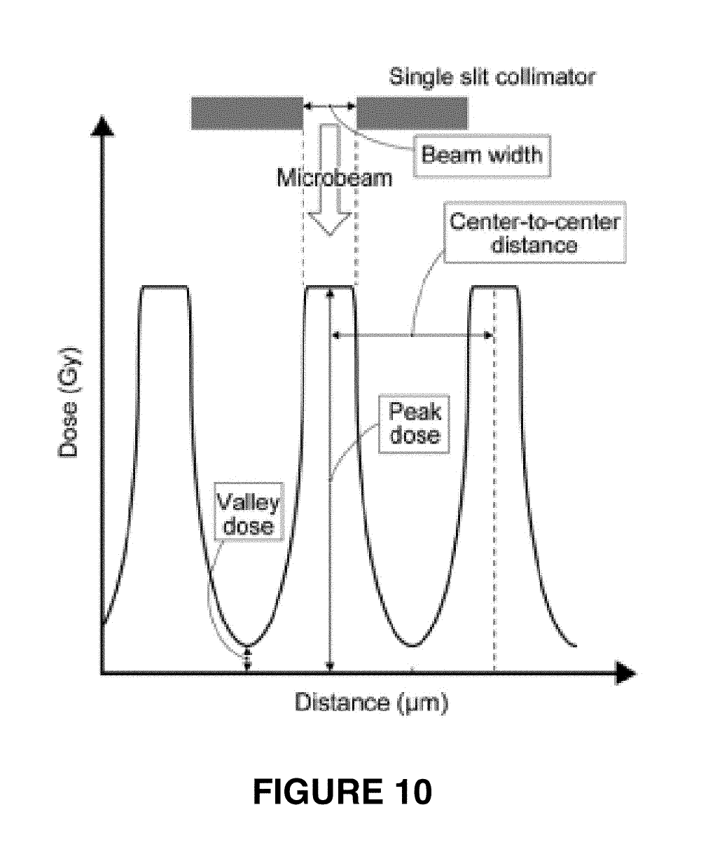

The selective destruction of Organic tissues, with/without the use of PS, the frequencies of X Rays, is determined by the dynamics of the secondary electrons and other charged particles, produced by the x-ray Beam (dashed arrow FIG. 2,3,4 Peaks, FIG. 10) to its Passage by the patient. In this mode the device and Generator x-ray Source (3) may not be moved.

In healthy tissue, St, and to preserve the dynamics of the secondary electrons, produced by the x-ray Beam is confined to the dimensions of the beam (dashed arrow in FIG. 2), given the dynamics of these electrons present in the tissue along the magnetic field Lines chaotic, LCC [5] where their Trajectories will be confined to the minimum expression, Minimizing the Biological Damage in ST.

In the target tissue (4), given the presence of the KIS (2), the charged particles produced by the beam, May be transmitted through, the intensity of Magnetic Field modulation and chaotic, and therefore the magnetic field intensity of KIS (2), and the Polarity of the same larger volumes, covering destruction of More controlled than in the current radiotherapy techniques, where there are no mechanisms control “partial”, Partial to do normal And because only those “Whose Control and Direction of the velocity Component perpendicular to the applied Magnetic Field possesses KIS (2), the Trajectories of the secondary electrons produced by the beam (dashed arrow in FIG. 2, The Peaks in FIGS. 10) to (4). For the Optimization of these mechanisms of partial control of the charged particles produced in (2) may be capable of Spinning/rotating Magnetic Field Generator (1), on the level of L2 (FIG. 1), to thereby vary the Topological structure and Direction of incidence of the magnetic field, KIS (2) to (4) applied in the x-ray Beam (dashed arrow in FIG. 2).

Sufficiently described the nature of the present Invention, as well as how to put it into practice, it is not necessary to do more extensive explanation for any expert in the Field, including its scope and the benefits derived therefrom, including, in its Essence, can be implemented in other forms realization that differ in Detail from the following example And which will also provide the protection that always not to alter, change or modify its fundamental principle.

### REFERENCES

[1] J. Aguirre, D. Peralta salas, “realistic examples of chaotic Magnetic Fields created by europhys wires”. Lett. 80, 60007, 2007.
[2] Gino Aguirre, J. J., and D. Peralta salas, “integrability of Magnetic Fields created by Current distributions nonlinearity 21, 51, 2008.
[3] Hosoda, M. T. Miyaguchi, K. however, and K. Nakamura, “Ubiquity of chaotic Magnetic Field Lines generated by three dimensionally crossed wires in Modern Electric Circuits,” Phys Rev e Stat nonlin Soft matter Phys. Dec 2009.
[4] T. Hosoda Miyaguchi, M., “topology of Magnetic Field Lines: Chaos and bifurcations emerging from two Action Systems”, Physical Review e 83, Jan 2011.
[5] J. Aguirre, Luque, D. PEAR to Salt as, “motion of charged particles in Magnetic Fields created by Symmetric configurations of wires”. Phys D, 239, May 10, 2010.
[6] Alejandro Luque and Daniel Peralta salas, “ABC motion of charged particles in Magnetic Fields”, Unpublished, DEP Barcelona University Applied Mathematics and analysis. April 2013.
[7] Ram, A. and B. Dasgupta, “Generation of chaotic Magnetic Fields and their effect on Particle Motion, EOS trans. Agu, 87 (52) Fall meet. Supplementum, ng31b - 1593, 2006.
[8] Ram, A. and B. Dasgupta, “Spatially Chaotic Dynamics of charged particles in Magnetic Fields”, Phys. Plasmas, 17, 122104.2010.
[9] E. Ott, Chaos in dynamical Systems, Cambridge University Press, Cambridge, 2002.
[10] Arnold Saw Kozlov, V V, neishtadt al Mathematical aspects of classical and Celestial Mechanics, dynamical Systems III, 3rd edn. Series: Encyclopaedia of Mathematical Sciences, Vol 3. Berlin, Springer, 2006
[11] Scholl E. and H. G. Schuster, Handbook of Chaos control, 2nd edwiley VCH, 2008.
[12] Graham Alan Webb, Modern Magnetic Resonance, Springer, 2008.
[13] Tomchuk P. M. and B. p. tomchuk, “Optical Absorption by Small Metallic particles”, ZH. EKSP. Teor. Fiz.112, 661 - 678, Aug 1997.
[14] Asenjo Garcia, Manjavacas et al., “magnetic polarization in the Optical Absorption of metallic Nanoparticles, Optics Express 28142, vol. 20, no. 27, Dec 2012.
[15] Shanghua Li, Meng Meng Lin, et al, “nanocomposites of Polymer and inorganic nanoparticles for Optical and Magnetic applications” nano Reviews: 5214 - doi: 1 10. Nano.v1i0.5214 3402/2010.
[16] Panikkanvalappil R. sajanlal, theruvakkattil s, et al, “anisotropic Nanomaterials: Growth, structure, Assembly, and functions”, nano 2 5883 - doi:: Reviews 10. 3402/nano.v2i0.58832011.
[17] Dev Kumar Chatterjee, parmeswaran diagaradjane, et al, “nanoparticle mediated hyperthermia in cancer therapy,” Ther deliv 1; 2 (8): 1001 - 1014. Aug 1 201.
[18] KK Jain, “Advances in the field of nanooncology BMC medicine2010,8: 83.
[19] S.s.r. Challa Kumar, Faruq Mohammad “magnetic Nanomaterials for hyperthermia based Therapy and controlled drug delivery” Advanced Drug Delivery Reviews (2011) - 63 789 808.
[20] Dykman L. N. G. khlebtsov, “Gold nanoparticles in Biology and Medicine: Recent advances and Prospects ”, Acta Naturae I vol. 3, no. 2 (9), 2011.
[21] C. Kramberger, H. Shiozawa, et al, “Anisotropy in the x-ray absorption of vertically aligned single wall carbon nanotubes. Phys. Stat. sol. (b) 244, no. 11, 1868 - 3981, 2007.
[22] Fischer, J. E. W. zhoum, et al, “magnetically aligned single wall carbon nanotube Films, Preferred orientation and anisotropic Transport Properties,” Journal of Applied Physics, Volume 93, Number 4, 2003.
[23] Slatkin D N et al. MicroBEAM radiation therapy“. Phys med 1992; 19: 1395. PubMed: 1461201 [].
[24] F.A. dilmanian et al, X - Ray microbeams: Tumor Therapy and central nervous system ”, nuci A. methods Phys Res 2005 August 11; 548 (1 - 2): 30-37.
[25] Atsushi Uyama et al, “a Narrow MicroBEAM for tumor growth suppression is more effective than a wide MicroBEAM: an in vivo study using implanted Human glioma cells”, J. Synchrotron Rad. (2011). 18 - 671 678.
[26] Gary Cook Jr. “Clinical nuclear medicine 4th edition” - Hodder Arnold Publication (2007).
[27] Berger, M. J. J. S. coursey, M. A. Zucker and J. Chang, available online: http:// Physics. NIST. Gov/Star, NIST, Gaithersburg, Md. (2005).
[28] Stefanescu, “Open Magnetic Field lines”, Rev. Roum. Phys.31701-21, 1986.
[29] John Sheffield, “The Physics of Magnetic Fusion Reactor” Oak Ridge National Laboratory, Oak, Tenn. 37831.
[30] T. E. Evans et al. “Edge Resonant Magnetic stability and Transport Control with
Perturbations in Tokamak plasmas collisionless Nature Physics “, June 2006, ucrl-jrnl-222189.
[31] J. A. Stratton 1941electromagnetic Theory (New York: McGraw Hill, 1986.
[32] Binhi V. N. magnetobiology (Academic Press, New York 2002).

1. Device for Selective Modification/destruction of Organic tissues, characterized in that it comprises: a Generator of chaotic Magnetic Fields (1) which has a Geometric configuration of Conductors so chaotic that generates a Magnetic Field, which is at least a Kam Torus Magnetic or Magnetic Island, located in a Topological Space (2), and that sets a Target tissue (4); a container that hosts the chaotic Magnetic Fields Generator (1) A Faraday Cage; (6) where it is housed in the container of the chaotic Magnetic Fields Generator; a solution of Nanoparticles biomolecules/destruction/modification introduced in the target tissue (4); one or several devices Generating electromagnetic waves (3, 3, 3 2), where the emission of electromagnetic waves is focused towards the target tissue (4) to modify/Destroy, the beam forming, Power, frequency, Consistency, and section suitable for Activation Resonant nanoparticles/biomolecules in the target tissue (4); and a room where you will stay the patient (5), Inside The Faraday Cage (6).

2. Device for Selective Modification/destruction of Organic tissues, according to claim 1, characterized by a RF Transmission and reception device (11) for the operation of nuclear magnetic resonance imaging; by a Lane that will mechanically Automated device and RF Transmission and reception (11) and a control panel (13) of the transmission and reception of RF coils (11) For Shaping the image of the target tissue (4) in MRI.

3. Device for Selective Modification/destruction of Organic tissues, according to claim 1, characterized by a estereotáxico device (8) for Stereotactic Localization of target Tissue to modify or destroy, and the intersection of the beam with the electromagnetic Magnetic Island Kam (4).

4. Device for Selective Modification/destruction of Organic tissues, according to claims 1, wherein the Generator device of electromagnetic frequencies (3.2) operate with do the frequencies of x-rays or gamma rays, without the application of Nanoparticles and biomolecules in the target tissue; the selective destruction of the target Tissue is produced by the interaction of the charged particles produced by the beam in the target Tissue and the Magnetic toroid (2) in the target tissue (4).

5. Device for Selective Modification/destruction of Organic tissues, according to claim 1 characterized, because the container that will hold the chaotic Magnetic Field Generator (1), for the cooling system of the coils, and on the basis of micrometric displacement Movement for the implementation of the definition of (4), and Volume to be treated; and can rotate on the central plane of the Cylinder (1) To enable changing the incidence of intersection between the Island Kam Magnetic electromagnetic Beam (2) and (4) the frequency of X Rays and gamma rays, to control the Trajectories of charged particles produced in the target tissue (4).

6. Device for Selective Modification/destruction of Organic tissues, according to claims 1, wherein the chaotic Magnetic Field Generating device (1) is connected to the Control Unit (7) from which they modify and control the electrical and Magnetic Parameters, the intensity and Voltage of the coils/terminal Drivers, while the unit, incorporated in a Booth outside the Faraday Cage (6).

7. A Biomedical Application using the device of claim 1.

8. The Biomedical Application of claim 7, wherein the device is used for the selective Modification of Organic tissues.

9. The Biomedical Application of claim 7, wherein the device is used for the selective destruction of Organic tissues.
